(19) United States
(12) Patent Application Publication
(10) Pub. No.: US 2020/0121852 A1
(43) Pub. Date: Apr. 23, 2020

(54) DEVICE AND METHOD FOR OPIOID OVERDOSE REVERSAL THROUGH RESPIRATION MONITORING AND AUTO-INJECTION OF MEDICATION FROM A WEARABLE DEVICE

(71) Applicant: James R. Winbush, Anderson, SC (US)

(72) Inventor: James R. Winbush, Anderson, SC (US)

(21) Appl. No.: 16/656,049

(22) Filed: Oct. 17, 2019

Related U.S. Application Data
(60) Provisional application No. 62/746,923, filed on Oct. 17, 2018.

(51) Int. Cl. A61M 5/20; A61B 5/11; A61B 5/145; A61B 5/00

(52) U.S. Cl. CPC A61M 5/20; A61B 5/1112; A61B 5/14542; A61B 5/681; A61M 2230/205; A61M 2205/3553; A61M 2210/083; A61M 2230/42; A61M 2005/206

(57) ABSTRACT

Method and adjustable body device worn on the body of a person, such as on their arm or wrist, for automatically administering pre-determined, regulated medication in the event the adjustable body device is activated by indication of respiratory distress such that the reversal of opioid overdose and respiratory depression from the use of opiate based medications is achieved.

### CROSS-REFERENCE TO RELATED APPLICATION

This application claims priority from U.S. Provisional Application Ser. No. 62/746,923 filed Oct. 17, 2018, the content of which is incorporated herein by reference in its entirety.

### BACKGROUND OF THE INVENTION

### 1. Field of the Invention

The present invention relates to the treatment of opioid overdose and, more specifically, to a device and method for reversing opioid overdose, including respiration monitoring and auto-injection of a medication to treat the opioid overdose.

### 2. Description of the Related Art

Opioid overdose can lead to decreased respiratory effort and death. The Center for Disease Control (CDC) currently estimates more than 1,000 emergency department visits related to the misuse of opioids occur daily and about 91 opioid overdose deaths occur every day, with the frequency of death rapidly increasing (1), as explained by Schiller, E Y., & Mechanic, O. J. “Opioid, overdose. StatPear/s ( ). Treasure Island (FL): StatPearls Publishing.

According to Schiller et al., Narcan® is a competitive antagonist of opiate receptors (1), and the administration of Narcan® can completely reverse the effects of opioid overdose and prevent deaths due to opioid overdose, based on information from an Information Sheet on Opioid Overdose generated by the World Health Organization (WHO).

### SUMMARY OF THE INVENTION

Disclosed is an adjustable body device worn on the body of a person, such as on their arm or wrist, which is used to automatically administer pre-determined, regulated medication in the event the device is activated by indication of respiratory distress. Preferably, the medication is an FDA approved medication, such as Naloxone hydrochloride or Narcan®.

In an effort to mitigate projected mortality rates due to opioid overdose, the device comprising an adjustable wrist or arm device with the pre-determined, regulated dosage of Narcan®, is worn and used as a way to prevent death in individuals experiencing respiratory distress as a result of opioid overdose. The device monitors respirations and O₂ (oxygen) saturation levels of an individual or patient wearing the device. Upon detection of respiratory depression, the device accordingly administers Narcan®.

At present, there are restrictions on morphine milli-equivalent amounts that can be administered to individuals or patients due to potential overdose. The reversal medication Narcan® will be mandatory in the near future, secondary to increasing deaths. Given these circumstances, the device and method of the invention has the potential to save millions of lives. Presently, in order to save an individual who has overdosed on an opioid, undergone respiratory distress, and is unconscious, another person is required to administer life-saving medication. Once a person is incapacitated as a result of respiratory distress, the patient is no longer capable of self-administering opioid reversal medication. In accordance with the invention, the device and method of the invention advantageously allow for the automation of this life saving measure.

With the device of the invention, it becomes possible for an individual to automatically receive opioid reversal medication without the assistance of someone else or through self-administration. Once a patient reaches a state of respiratory distress, the device in accordance with the invention injects life-saving opioid reversal medication automatically without any involvement from the patient or a bystander.

Opioid overdose causes death by respiratory depression. Respiratory depression is gradual, as there is a gradual decrease in the amount of respirations per minute. A normal respiratory rate can typically range from twelve to twenty breaths per minute. However, once respirations decrease to a rate of between three to four breaths per minute, respiratory distress ensues. The device in accordance with the invention monitors and detects the decrease in respirations as well as the O₂ saturation level of the patient and automatically administers Narcan® once the respiration rate falls to three to four breaths per minute. Here, the device is pre-loaded with a predetermined amount of Narcan®. In preferred embodiments, the device is pre-loaded with 0.4 mg/mL of Narcan®, which is the standard dose amount for intramuscular (IM) or subcutaneous (SC) Narcan® administration. The SC auto injection of Narcan® is administered through the use of a standard ⅜^{th} inch-⅝^{th} inch needle, whereas 1 to 1.5 inch needles are typically used for IM injection of Narcan®. In certain embodiments, the injection needle is located on the underside of the device to allow for optimal access to the patient (see FIG. 2), where the medication will be released via an auto-injector triggered by a signal from a microprocessor based on the qualifying criteria of respiratory distress occurring while the individual or patient is being monitored. One example of an auto-injector is manufactured by the company Evizo®. It should be understood, however, the Evizo® auto-injector is exemplary and other auto-injectors manufactured by other companies can be used.

After the device is triggered by the qualifying criteria of respiratory distress while it is monitoring the individual or patient, the subsequent release of Narcan® will enable GPS technology to immediately notify a local 911 dispatcher. This too is entirely automated and requires no involvement from the patient. The entire process is strictly governed by the monitored respiratory rate of the individual or patient. The administration of Narcan® through this novel and inventive approach has the potential to save lives by reversing opioid overdose and respiratory depression from the use of any opiate based medications.

In addition to opiate reversal, it is also contemplated to provide benzodiazipine reversal via the device and method of the invention. The recommended initial dose of flumazenil injection is 0.2 mg (2 mL) administered as a bolus, intramuscularly. If the desired level of consciousness is not obtained after waiting an additional 45 seconds, a second dose in the amount of 0.2 mg (2 mL) is injected and repeated at 60-second intervals where necessary (up to a maximum of 4 additional times) to a maximum total dose of 1 mg (10 mL). The dosage response, with most patients responding to doses of 0.6 mg to 1 mg (Individualization of Dosage).

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

An exemplary embodiment of the invention is explained in greater detail below with reference to the drawings, this comprising schematic and greatly simplified figures, in which:

FIG. 1 is an Illustration of the adjustable body device worn on the wrist of an individual in accordance with the invention;

FIG. 2 is a plan view of the adjustable body device in accordance with the invention;

FIG. 3 is an Illustration of the adjustable body device and medication size of the apparatus of FIG. 1; and

FIG. 4 is a flowchart of the method in accordance with the invention.

### DETAILED DESCRIPTION OF THE EXEMPLARY EMBODIMENTS

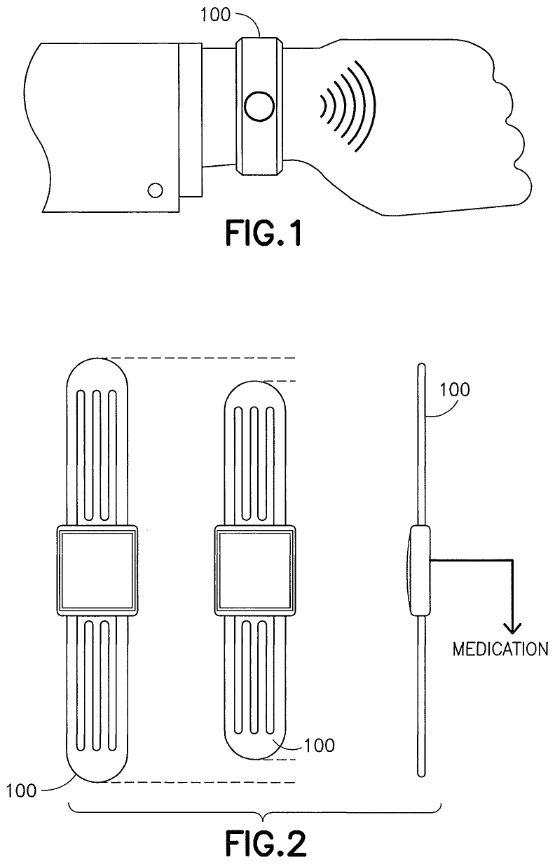

FIG. 1 is an illustration of an adjustable body device 100, which is worn on the body of a person, such as on their arm or wrist, and which is used to automatically administer pre-determined, regulated medication in the event the device is activated by the indication of respiratory distress. Preferably, the medication is an FDA approved medication, such as Naloxone hydrochloride or Narcan®.

In an effort to mitigate projected mortality rates due to opioid overdose, the device comprising an adjustable wrist or arm device with the pre-determined, regulated dosage of Narcan® is worn and used as a way to prevent death in individuals experiencing respiratory distress as a result of opioid overdose.

FIG. 2 is a plan view of the adjustable body device 100 illustrating the adjustable functionality in accordance with the invention. The features associated with the adjustment of a device worn on the arm or wrist of an individual are generally know, and therefore there will not be discussed in detailed here. What should be understood, however, is the pre-determined, regulated medication is delivered to the individual via a lower surface of the adjustable body device arm or wrist, as illustrated in the side view shown to the right in FIG. 2.

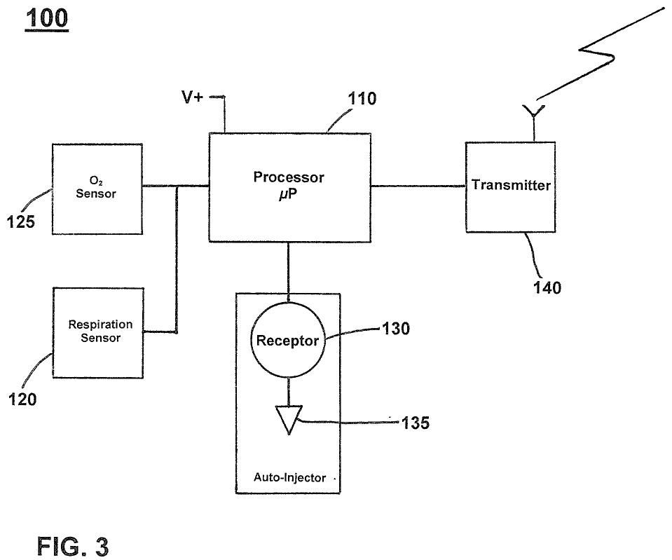

FIG. 3 is a schematic block diagram of an exemplary adjustable body device 100. The device 100 includes a microprocessor or processor 110 that performs calculations based on the monitored respiration rate of an individual or patient wearing the device 100 based on sensors 120 and 125, which provide the respiration and O₂ saturation levels of the individual, respectively.

A normal respiratory rate can typically range from twelve to twenty breaths per minute. However, once respirations decrease to three to four breaths per minute, respiratory distress ensues. The adjustable body device 100 in accordance with the invention monitors and detects the decrease in respirations as well as the O₂ saturation level of the patient and automatically administers Narcan® once the respiration rate falls to three to four breaths per minute. Here, the adjustable body device 100 is pre-loaded with a predetermined amount of Narcan® that is stored in a medication receptor 130. In preferred embodiments, the device is pre-loaded with 0.4 mg/mL of Narcan®, which is the standard dose amount for intramuscular (IM) or subcutaneous (SC) Narcan® administration. The SC auto injection of Narcan® is administered through the use of a standard needle 135, such as a ⅜^{th} inch-⅝^{th} inch needle, whereas 1 to 1.5 inch needles are typically used for IM injection of Narcan®. In certain embodiments, the injection needle 135 is located on the underside of the adjustable body device 100 to allow for optimal access to the patient (see FIG. 2), where the medication will be released via an auto-injector triggered by a signal from the microprocessor 110 based on the qualifying criteria of respiratory distress while the individual or patient is being monitored. One example of an auto-injector is manufactured by the company Evizo®. It should be understood, however, the Evizo® auto-injector is exemplary and other auto-injectors manufactured by other companies can be used.

After the adjustable body device 100 is triggered by the qualifying criteria of respiratory distress while it is monitoring the individual or patient, the subsequent release of Narcan® enables GPS technology, via a standard transmitter 140 connected to the microprocessor 110, to immediately notify a local 911 dispatcher. This too is entirely automated and requires no involvement from the patient, because the microprocessor 110 handles automation of the process. The entire process is strictly governed by the monitored respiratory rate of the individual or patient via the information obtained by the sensor 120 and provided to the microprocessor 110. The administration of Narcan® through this novel and inventive approach has the potential to save lives by reversing opioid overdose and respiratory depression from the use of any opiate based medications.

In addition to opiate based reversal, it is also contemplated to provide benzodiazipine based reversal via the adjustable body device 100 and method of the invention. The recommended initial dose of flumazenil injection is 0.2 mg (2 mL) administered as a bolus, intramuscularly. If the desired level of consciousness is not obtained after waiting an additional 45 seconds, a second dose of 0.2 mg (2 mL) is injected and repeated at 60-second intervals where necessary (up to a maximum of 4 additional times) to a maximum total dose of 1 mg (10 mL). The dosage response, with most patients responding to doses of 0.6 mg to 1 mg (Individualization of Dosage).

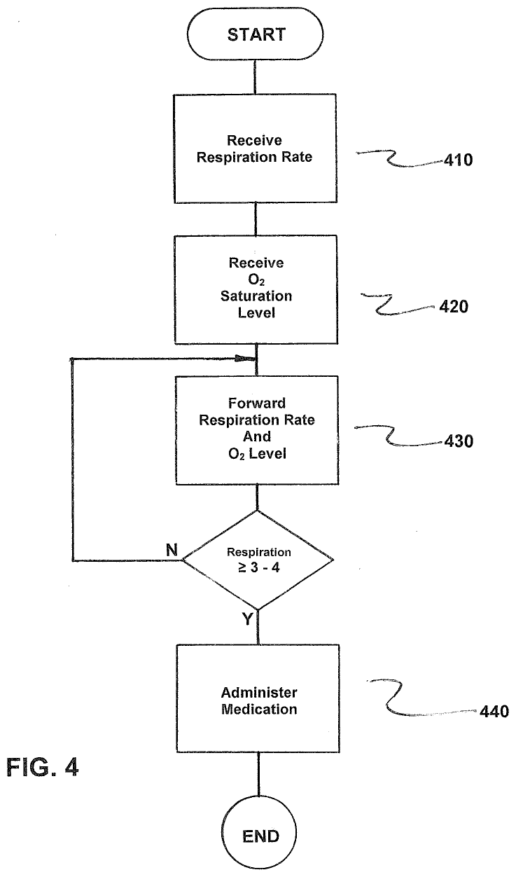

FIG. 4 is a flowchart of the method for reversing opioid overdose and respiratory depression via an adjustable body device 100. With reference to FIG. 4, the method comprises receiving a respiration rates of an individual wearing the adjustable body device 100 at a first sensor 120, as indicated in step 410. Next, an oxygen saturation level of the individual wearing the adjustable body device 100 is received at a second sensor 125, as indicated in step 420.

Next, the respiration rate and oxygen saturation level are forwarded from the first and second sensors 120, 125 to a microprocessor 110 and continually monitored at the microprocessor 110, as indicated in step 430.

Next, a pre-determined dosage of medication stored in an medication receptor is applied to the individual by a dosage applicator 130 based on an indication from the microprocessor 110 that the monitored respiration rate has reached a predetermined level, as indicated in step 440. A normal respiratory rate can typically range from twelve to twenty breaths per minute. However, once respirations decrease to three to four breaths per minute, respiratory distress ensues. In preferred embodiments, the device is pre-loaded with 0.4 mg/mL of Narcan®, which is the standard dose amount for intramuscular (IM) or subcutaneous (SC) Narcan® administration.

Thus, while there have been shown, described and pointed out fundamental novel features of the invention as applied to a preferred embodiment thereof, it will be understood that various omissions and substitutions and changes in the form and details of the methods described and the devices illustrated, and in their operation, may be made by those skilled in the art without departing from the spirit of the invention. For example, it is expressly intended that all combinations of those method steps which perform substantially the same function in substantially the same way to achieve the same results are within the scope of the invention. Moreover, it should be recognized that method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. An adjustable body device for reversing opioid overdose and respiratory depression, the adjustable body device comprising:
a first sensor which receives a respiration rates of an individual wearing the adjustable body device;
a second sensor which receives an oxygen saturation level the individual wearing the adjustable body device;
a microprocessor which receives and monitors the respiration rate and oxygen saturation level from the first and second sensors, respectively;
a medication receptor which stores a pre-determined dosage of medication; and
a dosage applicator which applies the pre-determined dosage of medication stored in the medication receptor based on an indication from the microprocessor that the monitored respiration rate reaches a predetermined level.

2. The adjustable body device of claim 1, further comprising:
a transmitter for providing location information to a Global Positioning System (GPS) upon triggering of the adjustable body device based on the indication from the microprocessor that the monitored respiration rate and oxygen saturation level reaches the predetermined level.

3. The adjustable body device of claim 1, wherein the predetermined level is three to four breaths per minute.

4. The adjustable body device of claim 1, wherein the medication comprises Narcan®.

5. The adjustable body device of claim 4, wherein the Narcan® is administrated intramuscularly (IM) or subcutaneously (SC).

6. The adjustable body device of claim 1, wherein the pre-determined dosage is 0.4 mg/mL of medication.

7. The adjustable body device of claim 1, wherein the dosage applicator is a needle within an auto-injector.

8. The adjustable body device of claim 7, wherein the needle of the auto-injector has a length of between ⅜^{th} to ⅝^{th} of an inch.

9. A method for reversing opioid overdose and respiratory depression via an adjustable body device, the method comprising:
receiving a respiration rate of an individual wearing the adjustable body device at a first sensor;
receiving an oxygen saturation level of the individual wearing the adjustable body device at a second sensor;
forwarding the received respiration rate and oxygen saturation level from the first and second sensors to a microprocessor for continual monitoring by the microprocessor; and
administering a pre-determined dosage of medication stored in an medication receptor to the individual via a dosage applicator within the adjustable body device based on an indication from the microprocessor that the continually monitored respiration rate has reached a predetermined level.

10. The method of claim 1, further comprising:
transmitting location information to a Global Positioning System (GPS) upon triggering of the dosage applicator within the adjustable body device based on the indication from the microprocessor that the monitored respiration rate and oxygen saturation level has reached the predetermined level.

11. The adjustable body device of claim 9, wherein the predetermined level is three to four breaths per minute.

12. The adjustable body device of claim 9, wherein the medication comprises Narcan®.

13. The adjustable body device of claim 12, wherein the Narcan® is administrated intramuscularly (IM) or subcutaneously (SC).

14. The adjustable body device of claim 9, wherein the pre-determined dosage is 0.4 mg/mL of medication.

15. The adjustable body device of claim 9, wherein the dosage applicator is a needle within an auto-injector.

16. The adjustable body device of claim 7, wherein the needle has a length of between ⅜^{th} to ⅝^{th} of an inch.
